(19) United States
(12) Patent Application Publication
(10) Pub. No.: US 2008/0274986 A1
(43) Pub. Date: Nov. 6, 2008

(54) PHARMACEUTICAL VEHICLE

(76) Inventor: Vanessa I. Chinea, San Antonio Aquadilla, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 12/174,596

(22) Filed: Jul. 16, 2008

Related U.S. Application Data
(62) Division of application No. 10/800,533, filed on Mar. 15, 2004.

(51) Int. Cl. A61K 31/7048; A61K 47/26; A61K 31/42; A61K 31/573; A61K 31/4375

(52) U.S. Cl. 514/26; 514/777; 514/380; 514/280; 514/177

(57) ABSTRACT

A pharmaceutical solution is capable of being ejected from a thermal fluid ejection device onto a substrate. The solution includes a vehicle with predetermined properties, and an active pharmaceutical ingredient with a solubility of at least about 30 mg/ml in the vehicle. The vehicle substantially evaporates from the substrate.

### CROSS-REFERENCE TO RELATED APPLICATIONS

This application is related to U.S. application Ser. No. 10/800,574, filed on Mar. 15, 2004, which is assigned to the assignee of the present application.

This application is also a divisional of co-pending U.S. application Ser. No. 10/800,533, filed Mar. 15, 2004, which is incorporated by reference herein in its entirety.

### BACKGROUND

Pharmaceutical doses in tablet or liquid form are made by pharmaceutical companies in formulations of a predetermined quantity of pharmaceutical units in each dose. Such pharmaceutical doses are frequently available in different strengths, such as 50 mg, 100 mg, etc.

A doctor typically prescribes a pharmaceutical or medication for a patient. The doctor, when prescribing a particular medication and medication strength, typically considers the patient's age, weight, sex, allergies, strong versus weak health condition, available dosage types, and the severity of the patient's illness, disease, or condition.

Especially for a low dosage, high potency drug, the pharmaceutical company may inadvertently form a pharmaceutical dose that is substantially lower than or that substantially exceeds the strength recommended by the doctor. Further, a pharmacist filling the prescription may inadvertently select the improper strength or the wrong pharmaceutical in filling the prescription. Additionally, the patient may take the incorrect pharmaceuticals, or take the proper pharmaceuticals at the wrong times, in the wrong amount, or in the wrong combination. If the concentration of the pharmaceutical falls under the prescribed therapeutic limit concentration for whatever reason, then the drug may lose efficacy. If the concentration exceeds a predetermined upper limit, the pharmaceutical may have toxic effects on the patient.

It would be desirable to provide a pharmaceutical dispensing apparatus and method which minimizes the occurrence of these issues, and provides a pharmaceutical dispensing apparatus and method which simplifies the manufacturing, distributing, and taking of pharmaceuticals.

### BRIEF DESCRIPTION OF THE DRAWINGS

FIG. 1 is a schematic block diagram of a pharmaceutical dispensing apparatus utilizing an embodiment of the method of the present invention.

FIG. 2 is a pictorial representation of a fluid dispenser according to an embodiment of the present apparatus.

FIG. 3 illustrates a perspective view of an embodiment of a fluid ejection cartridge of the present invention.

FIG. 4 is a perspective view of an embodiment of firing chamber of the cartridge of FIG. 3.

FIGS. 5A and 5B are cross-sectional views of an embodiment of the pharmaceutical medium.

### DETAILED DESCRIPTION

For the purposes of this description and the present invention, the term “pharmaceutical” is meant to include any type of drug, medication, chemical, or compound which is designed to be taken by a human as a medication to combat an illness or disease or to promote general health. Thus, pharmaceuticals as used herein, can be any drug, vitamin, or other chemical or compound which is used for health related purposes.

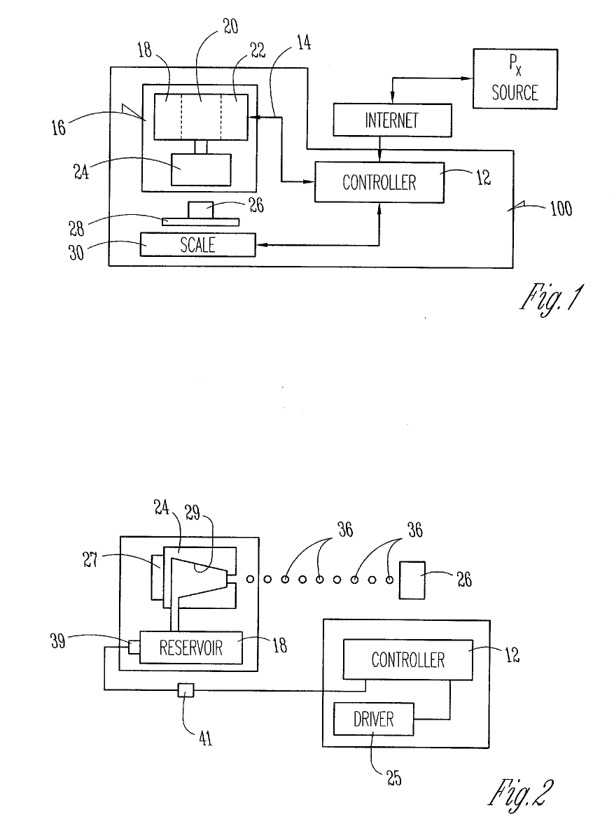

An embodiment of a pharmaceutical dispensing apparatus 100 of the present invention is shown in the schematic view of FIG. 1. The pharmaceutical dispensing apparatus 100 may be used in a pharmacy or other pharmaceutical dispensing location to automatically prepare custom pharmaceutical doses in response to prescription orders.

The pharmaceutical dispensing apparatus 100 includes a pharmaceutical medium 26, a fluid dispenser 16 to dispense a pharmaceutical solution onto the medium 26, and a controller 12 that receives information from, for example, a central processing unit, not shown, and then provides output signals 14 to the fluid dispenser 16. The pharmaceutical dispensing apparatus 100 further includes a media carrier 28 for the media 26, and a scale 30 to weigh the pharmaceuticals deposited onto the media 26.

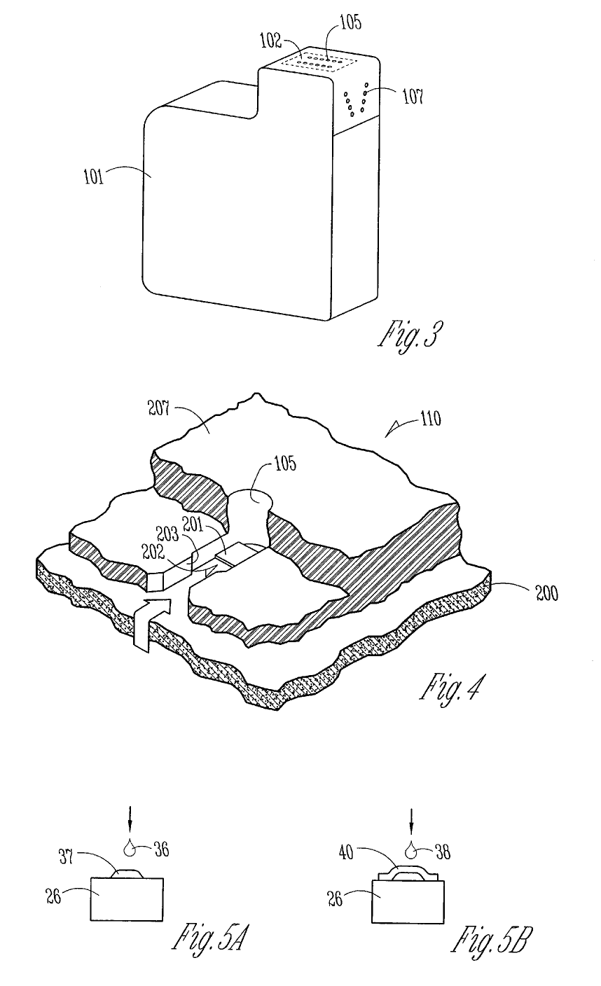

The fluid dispenser 16 includes an array 24 of fluid drop generators 110 (shown in FIG. 4) that eject a pharmaceutical solution. The pharmaceutical solution is often formed of an active pharmaceutical ingredient dissolved in a vehicle, including at least one solvent. The pharmaceuticals are dispensed to the pharmaceutical medium 26 for manufacturing a pharmaceutical dose for a patient or user. In one embodiment, the fluid dispenser enables the quantity of pharmaceutical(s) to be varied from dose to dose thereby enabling custom doses to be more easily prepared to appropriately suit each specific patient.

The fluid dispenser 16, which may incorporate control and structural features of Hewlett-Packard Ink-Jet printers, Model Nos. HP-C1823D and HP51645A, for example, includes at least one reservoir 18 which contains a quantity of a specific type of one pharmaceutical. In yet another embodiment, the reservoir 18 contains a mixture of several pharmaceuticals specific to a patient. In another embodiment, multiple reservoirs 20 and 22 are also provided in the fluid dispenser 16. Each reservoir 18, 20, and 22 is fluidically coupled to the array 24. Each reservoir 18, 20 and 22 may contain the same or different pharmaceuticals. In another embodiment, a single reservoir can contain a plurality of separate compartments.

In one embodiment, the reservoirs 18, 20, 22 are replaceable. The reservoir or reservoirs 18, 20, and 22 are fully charged with pharmaceutical components by the pharmaceutical manufacturer, for example. In one embodiment, the reservoirs are then shipped to the dispensing location, such as a pharmacy. In another embodiment, the fluid dispenser 16, including the reservoirs, is replaced and shipped. In another embodiment, the apparatus 100 is replaced when the pharmaceuticals in the reservoirs 18, 20, and 22 are exhausted, in that the apparatus 100 is exchanged for a completely new apparatus 100.

Multiple pharmaceuticals are typically taken by a user over the course of an illness or within a short time period. In one embodiment, multiple different pharmaceuticals from the reservoir(s) are enabled to be dispensed onto a single pharmaceutical receiving medium 26.

The controller 12 is capable of generating the control signals 14 which simultaneously or consecutively control the number of drops dispensed by the array 24 associated with one or more reservoirs, 18, 20, or 22.

Multiple identical pharmaceutical filled mediums 26 can be formed by the apparatus 100 with the same selected quantity and type of pharmaceuticals. In another embodiment, the apparatus 100 forms multiple different pharmaceutical filled mediums 26. The receiving medium 26 may be any suitable media used to receive, store, and transport pharmaceuticals. In one embodiment, the medium 26 is a two-dimensional substrate. In one embodiment, the medium 26 is an edible laminar substrate. In one embodiment, the medium 26 is a tablet. In one embodiment, a porous sugar tablet or even a liquid receiving vial may be employed as the medium 26.

The media carrier 28, such as a conveyor, not shown, may be employed to advance new, unfilled media 26 into proximity with the dispenser 16 as well as moving pharmaceutical filled media 26 away from the dispenser 16 and to a packaging or unload station, not shown.

The weight detector 30 of FIG. 1 may be any type of weighting device, such as an electronic scale, which is capable of measuring the weight of each receiving medium 26, both in an unfilled state and in a filled state. The output signals of the weight detector 30 are input to the controller 12. The controller 12 compares the measured weight of each filled medium 26 with a pre-stored, established reference or standard weight of a medium 26 and a complete quantity of a selected pharmaceutical to determine that the proper amount of pharmaceutical components have been dispensed to the medium 26. In an additional embodiment, the amount of dispensed pharmaceutical is measured using a spectrophotometric device.

The controller 12 can thus determine whether or not each medium 26 has been filled with the complete quantity of the selected pharmaceutical. If the detected weight comparison indicates that the medium 26 is too heavy, thereby indicating that too much pharmaceutical has been added to the medium 26, the controller 12 can activate a suitable reject apparatus, not shown, to reject the particular medium 26. Alternately, if the detected weight of the filled medium 26 is less than the standard or reference weight, the controller 12 can also generate signals activating the reject apparatus or, using feedback, determine the difference between the standard weight and measured weight of the medium 26 and then re-activate the array 24 to dispense a selected amount of the pharmaceutical(s) to bring the weight difference to zero.

In one embodiment of FIG. 1, an electrical communication device such as the Internet is provided with the controller 12 to receive signals from a remote signal source, such as a doctor's office or other prescription issuing authority. These signals contain, for example, patient identifying data, as well as the type(s) of pharmaceuticals, the quantity in terms of the number of dosage units to be made, the dosage strength, etc. These signals are input to the controller 12 which then activates the fluid dispenser 16 in the appropriate manner to prepare the specified pharmaceutical doses. This automated system minimizes errors in interpreting a doctor's handwritten prescription order as well as potential errors in manually filling the prescription.

In the embodiment of FIG. 1, the fluid dispenser 16 is a drop on demand type fluid dispenser. In one embodiment, the dispenser 16 has piezoelectric and/or thermal fluid drop generators. The embodiment depicted in the block diagram of FIG. 2 shows a typical piezoelectric array 24. Upon each activation of a piezoelectric driver 25 in the dispensing apparatus 100, pharmaceutical droplets 36 are dispensed to medium 26. The piezoelectric driver 25 operating under control signals from the controller 12 supplies activating signals to a disk or layer of piezoelectric material 27 which is mechanically connected to a chamber 29 in one of the drop generators of array 24. The chamber 29 is disposed in fluid communication with one of the reservoirs, such as reservoir 18, whereby capillary action supplies fluid pharmaceuticals from the reservoir 18 to the chamber 29. Upon each activation of the driver 25, the piezoelectric material 27 undergoes stress which results in mechanical movement of the piezoelectric material or element 27. This causes pumping action within the chamber 29 which expels individual droplets 36 through an orifice or outlet in the drop generator.

FIG. 2 also depicts a data or information storage device 39 which may be associated with each of the reservoirs 18, 20 and 22, with only reservoir 18 being shown. The storage device 39 is any type of memory device suitable for storing and outputting information related to parameters of the pharmaceutical contained within the particular reservoir 18 and/or the reservoir itself. The storage device 39 may be a memory chip mounted on the reservoir 18 and connected to external contacts which mate with contacts in a connector 41 when the reservoir 18 is mounted in the fluid dispenser 16 and connected electrically or optically with the controller 12. Once the connection between the contacts on the storage device 39 and the connector 41 is made, the controller 12 is disposed in electrical communication with the storage device 39 for information transfer with the storage device 39.

The information in the storage device 39 may be such as to enable the controller 12 to determine the type of pharmaceutical in the reservoir 18 as well as other information, such as the quantity of the pharmaceutical remaining the reservoir 18 based on the number of drops dispensed or the number of times that the array 24 has been activated. Other parameters which can be stored in the storage device 39 include a date code of manufacture of the pharmaceutical, an inspection date, system coefficients, reservoir size, and age of the pharmaceutical. The controller 12 can thereby verify that the proper pharmaceutical component is provided in the appropriate reservoir location or merely identify which pharmaceutical component is present. An example of a fluid dispenser having retrievable reservoir identification information is described in U.S. Pat. No. 6,039,430, assigned to the Assignee of the present invention.

One example of a thermal fluid dispenser 16 is the fluid ejection cartridge 101 shown in FIG. 3. The cartridge 101 has a fluid ejection device or fluid jet device 102. The cartridge houses the fluid supply, such as a pharmaceutical solution, in the reservoir. Visible at the outer surface of the ejection device are a plurality of orifices or nozzles 105 through which fluid is selectively expelled. In one embodiment, the fluid is expelled upon commands of the controller 12 communicated to the device 102 through electrical connections 107 (may also be wireless connections).

As shown in the perspective view of the embodiment illustrated in FIG. 4, the device 102 (or array 24) includes a plurality of fluid drop generators 110. In one embodiment, each drop generator 110 includes a thin film resistor 201 supported by a surface 200. A firing chamber 202 about each resistor 201 is formed by a nozzle layer 207. The nozzle chamber layer 207 also defines the orifice 105 corresponding to each resistor 201, and an entrance or fluid channel 203 to each firing chamber 202. Often, fluid flows through the fluid channel 203 into the firing chamber 202. Actuation of the heater resistor 201 by a “fire signal” causes fluid in the corresponding firing chamber 202 to be heated and expelled through the corresponding orifice 105.

In one embodiment, the pharmaceutical solution of the active ingredient dissolved in the vehicle is capable of being successfully fired from a thermal fluid ejection device. In one embodiment, the vehicle is stable at high ejection temperatures. In an additional embodiment, the solution is capable of withstanding the high temperatures generated in a thermal fluid ejection device. In another embodiment, at least one of the vehicle and the active pharmaceutical ingredient are not substantially exposed to high ejection temperatures. In this embodiment only a relatively small film of the vehicle is heated, which does not affect the pharmaceutical ingredient stability, nor the vehicle stability. In another embodiment, the solution is capable of being ejected from a piezoelectric ejection device.

In one embodiment, the solution including the vehicle and active ingredient have appropriate and predetermined fluidic properties for viscosity, density and surface tension such that the ejection from the ejection device is substantially successful and substantially repeatable in subsequent firings.

In one embodiment, the fluid ejection device 24 or 102 is formed of materials that are substantially inert to the pharmaceuticals which are to be dispensed therefrom, such as glass, ceramic, porcelain, and inert plastic. In another embodiment, the pharmaceutical solution is formed of components or substances that do not cause substantial corrosion or damage to the fluid ejection device or to the array. In one embodiment, the pharmaceutical solution does not substantially clog the nozzles of the array when the ejection device is in use under predetermined conditions.

In one embodiment, 2-pyrrolidone (2-P) is capable of being substantially successfully fired from the fluid ejection device. In one embodiment, dimethyl sulfoxide (DMSO) is capable of being substantially successfully fired from the fluid ejection device. In one embodiment, alcohols in general and ethanol in particular are both capable of being substantially successfully fired from the fluid ejection device. In an additional embodiment, additives to the pharmaceutical solution such as PVP, glycerine and PEG are capable of being substantially successfully fired from the fluid ejection device.

In one embodiment, the pharmaceutical solution is fired within a certain drop weight. In one embodiment, the drop weights are in a range of about 7 ng to about 140 ng. In another embodiment, the drop weight is not less than 8 ng. In one embodiment, the distance between drops is 1/600 of an inch upon the medium 26. In one embodiment from the fluid ejection device, pharmaceutical dosage drops are substantially reproducible with a reproducibility of less than about 15%.

In one embodiment, the fluid viscosity in the fluid ejection device is about 1.15 to about 1.44 cps. In one embodiment, the fluid viscosity in the fluid ejection device is about 2.6 to about 3.4 cps. In one embodiment, the fluid viscosity in the fluid ejection device is about 4.8 cps.

In one embodiment, the fluid surface tension is about 39-49 dynes/cm. In one embodiment, the fluid surface tension is about 46-54 dynes/cm. In one embodiment, the fluid surface tension is about 62 dynes/cm.

In one embodiment, the evaporation of the vehicle, such as a solvent, produces particles. After substantial evaporation of the vehicle, these particles may be present about the nozzles or on the substrate. In one embodiment, the smallest of these particles are about 21 microns in diameter and 5 microns in height. The particles are composed of crystals that are smaller than 21 microns. In one embodiment, the vehicle for the active ingredient evaporates about the nozzle, such that the particles remain about the nozzle, but these particles do not substantially clog the nozzle.

In one embodiment, the residue or particles that remain on the substrate after evaporation are edible and/or ingestible. In an additional embodiment, the vehicle used in transporting the active ingredient through the fluid ejection device is at least one of Generally Regarded as Safe, edible, ingestible, used in the pharmaceutical industry, and approved by the FDA. In one embodiment, each vehicle used in the solution has a low toxicity as listed in the ICH Topic Q3C Impurities: Residual Solvents (1997) at http://www.fda.gov/cber/gdlns/ich-g3ctablist.pdf and http://www.fda.gov/cber/gdlns/g3cresolvent.pdf. In another embodiment, inactive ingredients in approved drug products found on the FDA website at http://www.accessdata.fda.gov/scripts/cder/iig/inde-x.cfm are used as a vehicle.

In one embodiment, the Active Pharmaceutical Ingredient (API) includes a non-ionizable low-aqueous solubility drug. In an additional embodiment, the API includes at least one of Digoxin, prednisolone, sulfamethoxazole, and reserpine. In one embodiment, the API is soluble in a given solvent and is capable of being dispensed using TIJ technology. In another embodiment, a “bioactive agent” is dispensed in at least one of the fluid ejection device embodiments described herein. A “bioactive agent” refers to any type of drug, pharmaceutical, medication, medicament, vitamin, nutritional supplement, or another composition that is capable of affecting an animal, including humans.

In one embodiment, the vehicle into which the API is dissolved includes at least one solvent. In another embodiment, the vehicle includes a combination of organic solvents. In one embodiment, the vehicle includes at least one of 2-pyrrolidone (2-P), 1,2 hexanediol, sodium xylene sulfonate, ethylene glycol mono-phenyl ether, an alcohol, dimethyl sulfoxide (DMSO), n-methyl pyrrolidone (NMP), water and ethanol, hydroquinone, cyclodextrines, polyethylene glycol 400-600, absolute ethanol, propylene glycol, and glycerin. In one embodiment, the vehicle is an excipient or an exceptor that is an inert or slightly active substance used as a medium of administration for the API or medicinal agents.

In several embodiments, the vehicle may include 2-P or DMSO, and sometimes has co-solvents or additives as shown in the table below. In these several embodiments, the API includes digoxin or prednisolone. The solvents and co-solvents are listed by percentage of volume. The solubility is approximate.

| | | | Solubility |
|---|---|---|---|
| API | Solvent | Co-solvent | (mg/ml) |
| Prednisolone | DMSO 100% | | 240 (237-243) |
| Prednisolone | DMSO 90% | Methanol | 300 |
| | | 10% | |
| Prednisolone | DMSO 80% | Methanol | 300 |
| | | 20% | |
| Prednisolone | Methanol | | 26-43.6 |
| Prednisolone | Ethyl Alcohol | | 25-40 |
| Prednisolone | Benzyl alcohol | | |
| Prednisolone | Isopropyl alcohol | | At least 11.17 |
| Prednisolone | 2-P | | At least 18.7 |
| Prednisolone | Polyethylene glycols (PEGs) | | At least 14.8 |
| Prednisolone | Tetraethylene glycol | | At least 18.7 |
| Prednisolone | Ethanol 100% | | At least 25 |

| | | | | Solubility |
|---|---|---|---|---|
| API | Solvent | Co-solvent | Additive | (mg/ml) |
| Digoxin | Ethanol 100% | | | 9 |
| Digoxin | Ethanol 80% | Water 17% | Glycerin 3% | 9 |
| Digoxin | 2-P 100% | | | 102 (101.8) |
| Digoxin | 2-P 80% | Ethanol 20% | | At least 75 |
| Digoxin | 2-P 56% | Water 44% | | 56 |
| Digoxin | Acetone | | | Less than 30 |
| Digoxin | Ethyl acetate | | | Less than 16.3 |
| Digoxin | Glycerine | | | Less than 14.2 |
| Digoxin | Polyethylene | | | Less than 19.3 |
| | glycols (PEGs) | | | |
| Digoxin | 1,5-pentanediol | | | Less than 21.9 |
| Digoxin | Ethylene glycol | | | Less than 16.3 |
| | (LEG) | | | |
| Digoxin | Water | | | 0.024 to 0.07 |
| Digoxin | 55% Water | 45% H-107 | | 52 |
| Digoxin | Polyethylene | Water 1% | Propylene | .25 |
| | glycol 400 90% | | glycol 3% | |
| | Absolute ethanol | | | |
| | 6% | | | |
| Digoxin | DMSO 80% | (EtOH) | | 200 |
| | | ethanol | | |
| | | alcohol 20% | | |

In one embodiment, surfactants and other solubilizing agents (PVP) are used in the solution. The surfactants and other solubilizing agents, co-solvents, and/or additives may be used to improve the solubility of the API, the reliability of the ejection device, and/or the performance of the ejection device.

In one embodiment, the solubility of the active pharmaceutical ingredient is at least about 30 mg/ml in the vehicle. In an additional embodiment, the solubility of the active pharmaceutical ingredient in the vehicle is up to about 300 mg/ml. In one embodiment, the solubility of the API transported substantially successfully in the vehicle depends upon how efficiently the vehicle can be removed from the final dose, and the design of the final product such as firing 1M drops in an item that will be ingestible.

In one embodiment, solubility of the API in the solvent or vehicle is increased through at least one of the following ways: temperature changes, introducing multiple solutes and salting out, using complex ion formation, using solute pKa and/or solvent pH, altering the solute and solvent structure and/or polarity, using complexes and complexation, using hydroquinone with an active ingredient such as digoxin to increase absorption rate, and using a particular cyclodextrine with digoxin.

In one embodiment, content uniformity and percentage label claim is substantially obtained, especially in high potency and low dosage active ingredients. In one embodiment, a content uniformity with a reproducibility of less than about 15% is obtained for dosages of about 1.5 mg per dose of digoxin. In one embodiment, the concentration of the active pharmaceutical ingredient remains substantially constant from media to media with a deviation of less than about 15%.

In an embodiment of a method to evaluate reproducibility and repeatability of the pen, the following steps are taken:

(1) Solvent of DMSO:EtOH (ethanol alcohol) 80:20 (V/V) is used to prepare a solution of approx 200 mg/mL.

(2) A substrate of Teflon® coated aluminum film is coated with pure DMSO.

(3) 1st and 2nd fluid ejection devices (such as pens) are fired 3 times in several patterns upon the substrate.

(4) The substrate surface (or strip) is washed with Di water and UV analyzed using a spectrophotometer.

(5) The orifice plates of the respective pens are cleaned after 2 hours.

(6) The process is repeated.

Results from an embodiment of the evaluation method are given in the table below. Loading refers to the amount of active ingredient dispensed on top of the strip. The concentration variation refers to the concentration of the solution that is analyzed by the spectrophotometer. The analyzed solution is the dosage dispensed onto the strip dissolved in the water. A concentration of one (1) part per million is 1 mg/L. For example, 1.8 mg/sample refers to the amount of drug dispensed on top of the strip, and 1.8 ppm refers to the concentration of the solution that was analyzed by the spectrophotometer. RSD is relative standard deviation. All values are approximate.

| | | | RSD of | | |
|---|---|---|---|---|---|
| | | Concentration | analyzed | | RSD at |
| | Loading | variation | concentration | RSD at | time 2 |
| | (mg/sample) | (ppm) | (mg/sample) | time 0 | hrs |
| Pen 1 | 1.8 | 1.63 to 1.98 | 0.13 | 7.1% | 7.5% |
| Pen 2 | 1.6 | 1.25 to 1.78 | 0.1 | 5.7% | 8.2% |

In one embodiment using the fluid ejection device of the present invention, about 1 M of drops are used to prepare about a 0.125 mg dose tablet of digoxin with a concentration of about 3.4 mg/ml and a drop volume of about 30 pL per drop.

In FIG. 5A, a first at least one drop generator 110 has been activated by the controller 12 to dispense at least one fluid drop 36 of a first pharmaceutical solution onto the medium 26. The at least one fluid drop 36 is shown as being deposited on the surface of the receiving medium 26 in a layer 37. In another embodiment, the layer 37 is comprised of multiple fluid drops 36. In one embodiment, the layer 37 is a single active pharmaceutical ingredient. In another embodiment, the layer 37 includes multiple active pharmaceutical ingredients (either from a reservoir having more than one active ingredient, or from multiple reservoirs with different active ingredients). In one embodiment, the receiving medium 26 is formed of a porous material which will allow the fluid pharmaceutical to be absorbed into the interior of the medium 26.

In an additional embodiment shown in FIG. 5B, a second at least one drop generator 110 is activated by the controller 12 to dispense droplets 38 over the first dispensed pharmaceutical on the medium 26 to form layer 40. In one embodiment, this second at least one drop generator 110 corresponds with a different reservoir and/or different pharmaceutical solution than the first at least one drop generator activated in FIG. 5A. In one embodiment, layer 40 is a barrier material, such as a clear coat or other inert material which will not interact with the first dispensed pharmaceutical component layer 37. In one embodiment, layer 40 is a second pharmaceutical solution. In an additional embodiment, multiple layers are deposited over layer 40. These multiple layers can be of inert barrier materials or pharmaceutical ingredients.

It is therefore to be understood that this invention may be practiced otherwise than as specifically described. For example, embodiments of the present invention is not limited to the described thermally actuated fluid ejection devices, but may also include any thermally actuated fluid ejection device. In addition, embodiments of the present invention is not limited to thermally actuated fluid ejection devices, but may also include mechanically actuated printheads, as well as other fluid ejection devices.

Thus, the present embodiments of the invention should be considered in all respects as illustrative and not restrictive, the scope of the invention to be indicated by the appended claims rather than the foregoing description. Where the claims recite “a” or “a first” element of the equivalent thereof, such claims should be understood to include incorporation of one or more such elements, neither requiring nor excluding two or more such elements.

What is claimed is:

1. A method of dispensing a pharmaceutical, the method comprising:
supplying a plurality of fluid pharmaceutical components, each component in a reservoir;
fluidically coupling the reservoirs to at least one electronically controllable fluid drop generator; and
activating the fluid drop generator to eject variably selected quantities of the pharmaceutical components onto a solid, orally ingestible pharmaceutical receiving medium, wherein the fluid pharmaceutical components include a vehicle that substantially evaporates from the receiving medium, and an active pharmaceutical ingredient with a solubility of at least about 30 mg/ml in the vehicle;
wherein the vehicle is at least one selected from the group consisting of: 1,2 hexanediol, sodium xylene sulfonate, ethylene glycol mono-phenyl ether, an alcohol, dimethyl sulfoxide (DMSO), hydroquinone, a cyclodextrine, absolute ethanol, propylene glycol, water, ethanol, and glycerin.

2. The method of claim 1 wherein the vehicle contains a component that remains on the medium after evaporation, wherein the component has a low toxicity as listed in ICH Topic Q3C Impurities and is Generally Regarded as Safe.

3. The method of claim 1 wherein the solubility of the active pharmaceutical ingredient is up to about 300 mg/ml in the vehicle.

4. A method of producing pharmaceutical doses, comprising:
ejecting from a fluid ejection device a vehicle with predetermined properties together with an active pharmaceutical ingredient onto a substrate, wherein the vehicle substantially evaporates from the substrate;
wherein the vehicle is at least one selected from the group consisting of: 1,2 hexanediol, sodium xylene sulfonate, ethylene glycol mono-phenyl ether, an alcohol, dimethyl sulfoxide (DMSO), hydroquinone, a cyclodextrine, absolute ethanol, propylene glycol, water, ethanol, and glycerin.

5. The method of claim 4 wherein the predetermined properties of the vehicle comprise:
capability of being repeatedly ejected from the fluid ejection device with a predetermined level of performance;
a component that remains after evaporation that has a low toxicity as listed in ICH Topic Q3C Impurities; and
capability of allowing the active pharmaceutical ingredient to dissolve in the vehicle with a solubility of at least about 30 mg/ml.

6. A system for forming a pharmaceutical dose, comprising:
a vehicle for transporting an active pharmaceutical ingredient from a thermal fluid ejection device to a substrate;
wherein the vehicle for transporting substantially evaporates from the substrate,
wherein the active pharmaceutical ingredient has a solubility of at least about 30 mg/ml in the vehicle;
wherein the vehicle has a component that remains on the substrate after substantial evaporation;
wherein that component is Generally Regarded As Safe and is edible; and
wherein the vehicle includes at least one vehicle selected from the group consisting of: 1,2 hexanediol, sodium xylene sulfonate, ethylene glycol mono-phenyl ether, an alcohol, dimethyl sulfoxide (DMSO), hydroquinone, a cyclodextrine, absolute ethanol, propylene glycol, water, ethanol, and glycerin.

7. The system of claim 6 wherein the active pharmaceutical ingredient is at least one of a bioactive agent, Digoxin, a non-ionizable low aqueous solubility drug, prednisolone, sulfamethoxazole, reserpine, and any solid substance that is soluble in a given solvent and capable of being dispensed using TIJ technology.

8. The method of claim 6 wherein the vehicle is at least one of Generally Regarded as Safe, edible, ingestible, used in the pharmaceutical industry, approved by the FDA, stable at ejection temperatures, or capable of being ejected from the thermal fluid ejection device due at least in part to appropriate fluidic properties.
